(19) United States
(12) Patent Application Publication
(10) Pub. No.: US 2007/0265360 A1
(43) Pub. Date: Nov. 15, 2007

(54) Catalyst and Process for the Synthesis of C2-Oxygenates by the Hydrogenation of Carbon Monoxide

(75) Inventors: Hongyuan Luo, Dalian (CN); Yunjie Ding, Dalian (CN); Hongmei Yin, Dalian (CN)

Correspondence Address: NIXON & VANDERHYE, PC, 901 NORTH GLEBE ROAD, 11TH FLOOR, ARLINGTON, VA, 22203, US

(73) Assignee: DALIAN INSTITUTE OF CHEMICAL PHYSICS, Dalian (CN)

(21) Appl. No.: 11/630,361

(22) PCT Filed: Jun. 23, 2004
(86) PCT No.: PCT/GB04/02692

§ 371 Date: Jan. 30, 2007

(51) Int. Cl. B01J 21/06; B01D 53/02; C07C 29/00

(52) U.S. Cl. 518/716; 502/232

(57) ABSTRACT

A catalyst is invented for the synthesis of C₂-oxygenates by the hydrogenation of CO. The catalyst is composed of Rh—Mn—Fe-M₁-M₂/Si0₂, among them Mn, Fe, M₁ and M₂ and additives. M₁ can be Li or Na while M₂ can be Ru or Ir. The content of Rh is 0.1-3% by weight; the weight ratio of Mn/Rh is 0.5-12, the weight ratio of Fe/Rh is 0.01-0.5, the weight ratio of M₁/Rh is 0.01-1 and the weight ratio of M₂/Rh is 0.1-1.0. The catalyst is prepared by impregnation of the solution of corresponding compounds of each component in desired amount onto the carrier of Si0₂, which is followed by drying at 283-473 K. Before using, the catalyst is reduced by hydrogen or hydrogen-containing gas at 573-673 K for at least one hour after drying or after calcinations at 473-673 K for 2-20 h. These catalysts can convert CO and H₂ into ethanol, acetaldehyde, acetic acid and other C₂-oxygenates at a high conversion and a high selectivity under mild conditions.

This invention involves catalysts for the synthesis of C₂-oxygenates by the hydrogenation of CO. In more detail, it is about a multi-component catalyst based on rhodium for the hydrogenation of CO to produce ethanol, acetic acid, acetaldehyde and acetic ester.

This invention also involves the synthesis process of the catalysts and process of C₂-oxygenates synthesis from syngas under mild conditions.

With the decreasing oil resources in the world, increasing prices and consumption, the exploration of new energy resources become urgent worldwide. Among the C₂-oxygenates, ethanol becomes more and more important as high-octane number clean fuel and additive in gasoline. Therefore, the direct synthesis of ethanol from syngas attracts worldwide attention. In recent years, Rh-based catalysts with multi-promoters have been studied widely and many patents have been published. For instance, supported catalysts based on Rh—Fe in the patent GB1501891; the catalysts based on Rh—Mn, promoted with Mg or Ir and Li, in J6148437 and J62148438; the catalysts based on Rh—Mn—Ir—Li in the patent of J59227831; the catalysts based on Rh, Mn, Fe, Li in the patent of J6032733; and the catalysts based on Rh—Mn—Fe promoted by Li or Na. A common characteristic of the above catalysts is a high loading of Rh. Thus, the low time space productivity of C₂-oxygenates per unit rhodium and the high costs of catalyst synthesis limit the industrial applications of the catalysts.

The invention is to provide a catalyst and process for the synthesis of C₂-oxygenates by the hydrogenation of CO.

The other purpose of the invention is to provide a synthesis process for the catalysts.

The invented catalysts have low loading of rhodium, and high activity. The catalytic performance per weight unit of rhodium is very high. The invented catalyst is composed of Rh—Mn—Fe-M₁-M₂/SiO₂ among them M₁ is alkali metal elements such as Li or Na; M₂ is Ru or Ir. As stated in the invention, the weight loading of rhodium is 0.1-3%, preferably 0.3-2% and more preferably 0.7-1.5%. The weight ratio of Mn/Rh is 0.5-12, preferably 0.5-10 and more preferably 1-8. The weight loading of Fe is 0.01-0.5, preferably 0.02-0.3, and more preferably 0.04-0.2. The weight ratio of M₁/Rh is 0.01-1, preferably 0.02-0.5, and more preferably 0.04-0.2. The weight ratio of M₂/Rh is 0.1-1.0, preferably 0.2-0.8 and more preferably 0.3-0.7. According to a preferred embodiment of the present invention, the invented catalyst does not comprise additives like Ag and/or Zr.

The preparation process for the catalysts is described as follows:

The catalysts are prepared by the impregnation method. The preferable method would be co-impregnation, but stepwise impregnation is also possible. The precursors for the components in the catalysts can be chlorides, nitrates or other dissolvable compounds, for instance, ammonia coordinated chlorides, carbonyl group coordinated and etc. The solvents can be water, or non aqueous solvent such as methanol.

When the co-impregnation method is used to prepare the catalysts, the precursor compounds are dissolved into a solvent. Then the solution with a certain concentration is impregnated onto the silica gel support. A minimum amount of the impregnation solution is required to submerge all support of the silica gel. When the method of the step-wise impregnation is used, the corresponding compounds are made into solutions with certain concentrations, these solutions are impregnated onto the catalyst support of the silica gel stepwise, or several compounds are made into a mixture solution, which is impregnated prior to the rest solutions of corresponding compounds.

The drying temperature is 283-473 K, with the drying time of 2 h to 20 days. The drying time is related to the drying temperature chosen. When the drying temperature is 373-393 K, the drying procedure can last 4-12 h. The dried catalyst can be calcined at 473-673 K for 2-20 h, but it can also be used as catalyst precursor directly. This catalyst precursor needs to be reduced in pure hydrogen or hydrogen-containing gas. The invented catalysts show a high space time yield for the C₂-oxygenates.

The catalysts for the C₂-oxygenates synthesis from syngas are first activated in-situ in a H₂ flow at SV=100-5000 h⁻¹, preferably 500-2000 h⁻¹; T=500-750 K, preferably 573-673 K; P=0.1 to 1.0 MPa, preferably 0.1 to 0.5 MPa.

The process for the C₂-oxygenates synthesis from syngas using above Rh based catalysts are carried out under following conditions: T=473-723 K, preferably 473-623 K; P=1.0-12.0 MPa, preferably 2.0-8.0 MPa; volume ratio of H₂/CO=1.0-3.0, preferably 2.0-2.5; space velocity−1000-50000 h⁻¹; preferably 10000-25000 h⁻¹.

### EXAMPLES

### Example 1

### The Preparation Process for the Catalysts

The silica support is impregnated by a certain amount of an aqueous solution of RhCl₃.xH₂O, Mn(NO₃)₂, LiNO₃, Fe(NO₃)₂, H₂IrCl₆, which is followed by drying at 383 K for 6 h. The obtained catalyst has a chemical composition 1% Rh=1% Mn-0.05% Fe-0.075% Li-0.5% Ir/SiO₂ (weight ratio).

### Example 2

### The Synthesis Process for the Catalysts

The silica support is impregnated by a certain amount of an aqueous solution of RhCl₃.xH₂O, Mn(NO₃)₂, LiNO₃, Fe(NO₃)₂, H₂IrCl₆ and dried at 383 K for 6 h. Thus a catalyst of 1% Rh-1% Mn-0.1% Fe-0.075% Li-0.5% Ir/SiO₂ is obtained (weight ratio).

### Example 3

The silica supported is impregnated by a certain amount of an aqueous solution of RhCl₃.xH₂O, Mn(O₃)₂, LiNO₃, Fe(NO₃)₂, H₂IrCl₆ and dried at 383 K for 6 h. Thus a catalyst of 1% Rh-1% Mn-0.05% Fe-0.1% Li-0.5% Ir/SiO₂ is obtained (weight ratio).

### Example 4

The silica support is impregnated by a certain amount of an aqueous solution of PhCl₃.xH₂O, Mn(NO₃)₂, NaNO₃, Fe(NO₃)₂, H₂IrCl₆ and dried at 383 K for 6 h. Thus a catalyst of 1% Rh-1% Mn-0.05% Fe-0.1% Na-0.5% Ir/SiO₂ is obtained (weight ratio).

### Example 5

The silica support is impregnated by a certain amount of an aqueous solution of RhCl₃.xH₂O, Mn(NO₃)₂, LiNO₃ Fe(NO₃)₂, RuCl₃ and dried at 383 K for 6 h. Thus a catalyst of 1% Rh-1% Mn-0.1% Fe-0.075% Li-0.5% Ru/SiO₂ is obtained (weight ratio).

### Example 6

The silica support is impregnated by a certain amount of an aqueous solution of RhCl₃.xH₂O, Mn(NO₃)₂, NaNO₃, Fe(NO₃)₂, RuCl₃ and dried at 383 K for 6 h. Thus a catalyst of 1% Rh-2% Mn-0.05% Fe-0.1% Na-0.5% Ru/SiO₂ is obtained (weight ratio).

### Example 7

The silica support is impregnated by a certain amount of an aqueous solution of RhCl₃.xH₂O, Mn(NO₃)₂, LiNO₃, Fe(O₃)₂, H₂IrCl₆ and dried at 383 K for 6 h. Thus a catalyst of 1.5% Rh-1.5% Mn-0.12% Fe-0.11% Li-0.5% Ir/SiO₂ is obtained (weight ratio).

### Comparison Example 1

The silica support is impregnated by a certain amount of an aqueous solution of RhCl₃.xH₂O, which is followed by drying at 383 K for 6 h. The obtained catalyst consists of 1% Rh/SiO₂ (weight ratio).

### Comparison Example 2

The silica support is impregnated by a certain amount of an aqueous solution of RhCl₃.xH₂O and Mn(NO₃)₂, followed by drying at 383 K for 6 h. Thus a catalyst of 1% Rh-1% Mn/SiO₂ is obtained (weight ratio).

### Comparison Example 3

The silica support is impregnated by a certain amount of an aqueous solution of RhCl₃.xH₂O, Mn(NO₃)₂, LiNO₃, H₂IrCl₆, which is followed by drying at 383 K for 6 h. The obtained catalyst has a chemicals composition 1% Rb-1% Mn-0.075% Li-0.5% Ir/SiO₂ (weight ratio)

### Comparison Example 4

The silica support is impregnated by a certain amount of an aqueous solution of RhCl₃.xH₂O, Mn(NO₃)₂, Fe(NO₃)₂, followed by drying at 383 K for 6 h. The obtained catalyst has a chemical composition 1% Rh-1% Mn-0.05% Fe/SiO₂ (weight ratio).

### Comparison Example 5

The silica support is impregnated by a certain amount of an aqueous solutions of RhCl₃.xH₂O, Mn(NO₃)₂, LiNO₃, H₂IrCl₆, which is followed by drying at 383 K for 6 h. The obtained catalyst has a chemical composition 1% Rh-1% Mn-0.075% Li-0.5% Ir/SiO₂ (weight ratio).

A series of comparative performance tests were conducted with 0.4 grams (˜0.8 ml) samples of the catalysts (20-40 mesh) from the Examples. The testing apparatus consisted of a small fixed bed tubular reactor with an external heating system, which was made of 316 L stainless steel with 340 mm length, 4.6 mm inner diameter. The catalyst was in-situ reduced in a flow of H₂ before test. The temperature was raised at 2 K/min from room temperature up to 623 K, and then held at constant for 1 h. The H₂ flow rate was 41/h at atmosphere pressure. Then the catalyst was shifted into syngas (H₂/CO=2) after cooling down to 523 K, and reacted under process conditions of T=593 K, P=3.0 MPa, SV=13000 h⁻¹ for 4 h. The effluent passed through a condenser filled with 150 ml deionised water which capture the oxygenates products. The aqueous solution containing oxygenates obtained was analysed off-line by Varian CP-3800 gas chromatography with an FEAP column, using FID detector and 1-pentanol as an internal standard. The tail gas was on-line analysed by Varian CP-3800 GC with a Porapak QS column and TCD detector.

The catalytic performances of the example catalysts and the comparison example catalysts are listed in Table 1.

The results in Table show that the activity and selectivity of the example catalysts for the synthesis of ethanol, acetic acid and acetaldehyde are higher although the loading of rhodium is lower and the catalyst synthesis process is simple. The rhodium efficiency of the example catalysts is obviously higher than the comparison example catalysts, which is promising for the industrial applications.

TABLE 1
Comparison of the catalytic performance of the example catalysts
and the comparison example catalysts*

| | The chemical composition | Time-space | |
|---|---|---|---|
| | of the catalyst (weight %, | yield | Selectivity |
| Catalyst | the rest Si02) | g/kg · h | C % |
| Example 1 | 1% Rh—1% Mn—0.075% Li—0.5% Ir—0.05% Fe | 460.2 | 62.2 |
| Example 2 | 1% Rh—1% Mn—0.075% Li—0.5% Ir—0.1% Fe | 453.1 | 60.4 |
| Example 3 | 1% Rh—1% Mn—0.1% Li—0.5% Ir—0.05% Fe | 445.7 | 60.1 |
| Example 4 | 1% Rh—1% Mn—0.1% Na—0.5% Ir—0.05% Fe | 438.3 | 60.4 |
| Example 5 | 1% Rh—1% Mn—0.075% Li—0.5% Ru—0.1% Fe | 428.6 | 58.3 |
| Example 6 | 1% Rh—2% Mn—0.1% Na—0.5% Ru—0.05% Fe | 432.7 | 57.4 |
| Example 7 | 1.5% Rh—1.5% Mn—0.075% Li—0.5% Ir—0.1% Fe | 509.1 | 64.3 |
| C example 1 | 1% Rh | 35.5 | 16.3 |
| C example 2 | 1% Rh—1% Mn | 270.3 | 34.9 |
| C example 3 | 1% Rh—1% Mn—0.075% Li | 331.6 | 56.8 |
| C example 4 | 1% Rh—1% Mn—0.05% Fe | 333.3 | 40.0 |
| C example 5 | 1% Rh—1% Mn—0.075% Li—0.5% Ir | 397.5 | 59.0 |

*The reaction conditions: H₂/CO = 2 (volume ratio), pressure 3.0 MPa; temperature 583 K; the space velocity (volume) 13000 h⁻¹.

1. Catalyst for the synthesis of C₂-oxygenates by the hydrogenation of CO consisting of components Rh—Mn—Fe-M₁-M₂ supported on silica wherein M₁ can be Li and/or Na and M₂ can be Ru and/or Ir, wherein Rh is 0.1 to 3% by weight based on the total catalyst weight and
the weight ratio of Mn/Rh: 0.5-12,
the weight ratio of Fe/Rh: 0.01-0.5,
the weight ratio of M₁/Rh: 0.01-1,
the weight ratio of M₂/Rh: 0.1-1.0.

2. Process for the preparation of a catalyst according to claim 1 comprising the preparation of a solution obtained by dissolving the compounds of the corresponding components of desired amount in solvents, impregnating the solution onto the silica gel catalyst support, which is followed by drying at 283-473 K for 2 h-20 days.

3. Process according to claim 2 wherein the compounds used are dissolvable chlorides or nitrates and the solvents are water or non-aqueous solvents.

4. Process according to claim 2 wherein the silica gel is produced by sol process and then heated in a basic solution, followed by drying and/or calcinating.

5. Process according to claim 2 wherein the compounds used are ammonia coordinated chlorides or carbonyl group coordinated compounds.

6. Process according to claim 2 wherein the solvent is methanol.

7. Process according to claim 2 wherein the catalyst is reduced in-situ in pure hydrogen or hydrogen containing gas at 573-673 K for at least on hour.

8. Process according to claim 2 wherein the impregnation is done either by co-impregnation or stepwise impregnation of all components.

9. Process as claimed in claim 8 wherein the stepwise impregnation is carried out at an arbitrary sequence.

10. Catalyst for the synthesis of C₂-oxygenates obtainable by a process according to claim 2.

11. Use of a catalyst according to claim 1 for the synthesis of C₂-oxygenates from synthesis gas.

12. Use according to claim 11 wherein the oxygenates are mainly ethanol, acetaldehyde and acetic acid.
